(19) United States
(12) Patent Application Publication
(10) Pub. No.: US 2022/0011146 A1
(43) Pub. Date: Jan. 13, 2022

(54) Airway Detection Using Air Pressure Differential

(71) Applicant: Avent, Inc., Alpharetta, GA (US)

(72) Inventor: James F. Tassitano, Marietta, GA (US)

(21) Appl. No.: 16/925,630

(22) Filed: Jul. 10, 2020

(51) Int. Cl. G01F 1/88; A61M 16/00

(52) U.S. Cl. CPC G01F 1/88; A61M 2016/0036; A61M 2016/0027; A61M 16/0003

(57) ABSTRACT

A medical device position notification system that includes a differential air pressure sensor is provided. The differential air pressure sensor may be attached to a medical device, e.g. a catheter, that is configured to be inserted into a patient's body. The differential air pressure sensor is configured to alert in real time when at least a portion of the medical device is in the patient's trachea or airway versus the esophagus or gastrointestinal tract. A method for medical device position guidance using a medical device position notification system that includes a differential air pressure sensor is also provided.

### FIELD OF THE INVENTION

The subject matter of the present invention relates generally to a system and method for detection placement of a medical device in the airway by detecting the air pressure differential.

### BACKGROUND

Physicians and other health care providers frequently use catheters to treat patients. The known catheters include a tube which is inserted into the human body. Certain catheters are inserted through the patient's nose or mouth for treating the gastrointestinal tract. These catheters, sometimes known as enteral catheters, typically include feeding tubes. The feeding tube lies in the stomach or intestines, and a feeding bag delivers liquid nutrient, liquid medicine or a combination of the two to the patient.

When using these known enteral catheters, it is important to place the end of the catheter at the proper location within the human body. Erroneous placement of the catheter tip may injure or harm the patient. For example, if the health care provider erroneously places an enteral catheter into the patient's trachea, lungs, or other regions of the respiratory system rather than through the esophagus and to the stomach to reach the desired location in the digestive tract for delivering nutrients or medicine, liquid may be introduced into the lungs with harmful, and even fatal, consequences. In particular, the esophagus of the digestive tract and the trachea of the respiratory system are in close proximity to each other and are blind to the health care provider during catheter placement, which creates a dangerous risk for erroneous catheter placement.

In some cases, health care providers use X-ray machines to gather information about the location of catheters within the body. There are several disadvantages with using X-ray machines. For example, these machines are relatively large and heavy, consume a relatively large amount of energy and expose the patient to a relatively high degree of X-ray radiation. Also, these machines are typically not readily accessible for use because, due to their size, they are usually installed in a special X-ray room. This room can be far away from the patient's room. Therefore, health care providers can find it inconvenient to use these machines for performing catheter insertion procedures. Moreover, even X-rays are not necessarily conclusive as to the location of the catheter tip, as the natural and continuous movement of the internal organs can make it difficult for the physician interpreting the X-ray to be sure of the actual location of the distal end of the catheter. In addition, using X-ray technology is expensive and is a time-consuming task that can create unnecessary delays in delivering critical nutrients to the patient.

Another existing catheter locating means involves using an electromagnetic coil positioned inside the catheter and an electromagnetic coil locating receiver outside of the patient's body. The electromagnetic coil is generally incorporated into a stylet or guide wire which is inserted within the catheter. The coil locating receiver can be used to determine the distance the coil is from the receiver and its depth in the patient's body and can communicate with a display to show a reference image of a non-subject body and an image of the coil located on the display with the reference image. However, these systems also have several disadvantages. For example, the coil locating receiver is a large device that must rest in a precise location outside the patient's body and does not permit for adjustments due to each individual patient's anatomical size or shape. However, a patient undergoing a feeding tube placement will be agitated and sudden movements are expected, which can move the coil locating receiver, thus increasing the likelihood of positional errors or complications in locating the catheter. Additionally, these existing systems can only display the coil location over a reference image of a non-subject (i.e., a generic patient) body without reference to the individual patient's particular anatomy. Thus, these existing systems can only generate generic warnings or alerts when a deviation from an intended path within the body is estimated. Such generic warnings or alerts are easily ignored by a health care provider because they provide little specific, actual information regarding the position of the catheter and do not adequately capture a health care provider's attention. Therefore, health care providers can estimate the positioning of the catheter using the electromagnetic coil and coil locating receiver but cannot estimate or view the specific patient's anatomy.

Consequently, there is a need for a system for notifying a user of the positioning of a medical device within a patient's body in real-time to ensure more accurate catheter placement. In particular, a notification system that is easy to use and provides a clear deviation alert when the medical device is improperly positioned would also be useful.

### SUMMARY

Objects and advantages of the invention will be set forth in part in the following description, or may be obvious from the description, or may be learned through practice of the invention.

The present invention is directed to a medical device position notification system. The system includes a medical device, wherein at least a portion of the medical device is configured to be inserted into a patient's body; and a differential air pressure sensor, wherein the differential air pressure sensor is configured to provide information relating to the position of the medical device in the patient's body.

In one particular embodiment, the differential air pressure sensor can be configured to measure air pressure within the medical device compared to ambient atmospheric air pressure. Further, when the differential air pressure measured over time matches a pattern of inhalation and exhalation, the medical device may be in the trachea or respiratory tract of the patient's body. Moreover, when the differential air pressure measured over time does not match a pattern of inhalation and exhalation, the medical device may be in the esophagus or gastrointestinal tract of the patient's body.

In one embodiment, the differential air pressure sensor can include a first port for receiving air flow from the medical device and a second port for receiving air flow from ambient air.

In one embodiment, the differential air pressure sensor can be configured to be electrically connected to at least one processor, wherein the differential air pressure sensor can measure information relating to the position of the medical device within the patient's body and can send signals containing the information relating to the position of the medical device within the patient's body to the processor via a wired or wireless electrical connection in real-time, further wherein a display device can be coupled to the processor and can display information relating to the position of the medical device within the patient's body communicated by the differential air pressure sensor.

In one embodiment, the medical device can include a catheter. Further, the catheter can be configured to be inserted into at least one orifice the patient's body. Moreover, the at least one orifice can include a nose or a mouth. Further, the differential air pressure sensor can be coupled to the catheter via a connector.

The present invention is further directed to a method for medical device position guidance. The method includes steps of: providing a medical device, wherein at least a portion of the medical device is configured to be inserted into the body; providing a differential air pressure sensor, wherein the differential air pressure sensor is configured to be coupled to the medical device; inserting the medical device into an orifice of the body; activating the differential air pressure sensor to sense air pressure in the medical device compared to ambient atmospheric air pressure; and observing the differential air pressure sensor or a display device configured to be coupled the differential air pressure sensor to determine the position of the medical device within the patient's body.

In one particular embodiment, the orifice can be a nose or a mouth.

In one embodiment, the differential air pressure sensor can be configured to measure the air pressure within the medical device compared to the ambient atmospheric air pressure. Further, the observing step can include observing the differential air pressure within the medical device over time, wherein if a pattern indicating inhalation and exhalation is observed, the medical device is in the trachea or respiratory tract of the patient's body.

In one embodiment, the observing step can include observing the differential air pressure within the medical device over time, wherein if a pattern indicating inhalation and exhalation is not observed, the medical device is in the esophagus or gastrointestinal tract of the patient's body.

In one embodiment, the differential air pressure sensor can be configured to be electrically connected to at least one processor, wherein the at least one differential air pressure sensor receives information relating to the position of the medical device within the patient's body and sends signals containing the information relating to the position of the medical device within the patient's body to the processor via a wired or wireless electrical connection in real-time, further wherein the display device is coupled to the processor and displays information relating to the position of the medical device within the patient's body communicated by the differential air pressure sensor.

In one embodiment, the medical device can include a catheter. Further, the differential air pressure sensor can be coupled to the catheter via a connector.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of a medical device position notification system in the form of an enteral catheter according to one embodiment of the present invention;

FIG. 2 illustrates a perspective view of a medical device position notification system according to various embodiments of the present invention;

FIG. 3 illustrates a perspective view of a differential air pressure sensor system according to various embodiments of the present invention;

FIG. 4 illustrates a perspective view of a medical device position notification system in the form of an enteral catheter according to various embodiments of the present invention;

FIG. 5 illustrates a perspective view of a graph generated by a medical device position notification system according to various embodiments of the present invention showing differential air pressure when the medical device is positioned in the airway of a patient; and

FIG. 6 illustrates a perspective view of a graph generated by a medical device position notification system according to various embodiments of the present invention showing differential air pressure when the medical device is positioned in the digestive tract of a patient.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

The medical device position notification system of the present disclosure is based on differential air pressure methodology and may be utilized to detect medical device placement, i.e. nasogastric or nasojejunal tube placement, in the airway without the need for cumbersome equipment, electronics, or complicated techniques. Indeed, the effectiveness of the disclosure of the medical device position notification system herein is predicated on anatomical differences between the esophagus and the trachea. For example, the air pressure within the airway, e.g., trachea, rises and falls in relation to ambient atmospheric pressure when air is passing in and out of the lungs during respiration. Conversely, the differential air pressure in the esophagus does not fluctuate up or down with respiration because there is no air passing in and out of the esophagus. Thus, observing the differential air pressure within the tube, including through the inner lumen of the nasogastric/nasojejunal tube, in relation to ambient atmospheric air pressure during placement can differentiate the location of the catheter or tube's tip based on this anatomical difference.

Generally speaking, the present disclosure is directed to a medical device position notification system that includes a differential air pressure sensor. The differential air pressure sensor may be attached to a medical device, e.g. a catheter, that is configured to be inserted into a patient's body. The differential air pressure sensor is configured to alert in real time when at least a portion of the medical device is in the patient's trachea or airway versus the esophagus or gastrointestinal tract. The present inventors have found that the medical device position notification system and method(s) described in more detail herein are easy to use and provide a real time information and signaling concerning the placement of a medical device, such as a catheter or enteral catheter, within a patient's body.

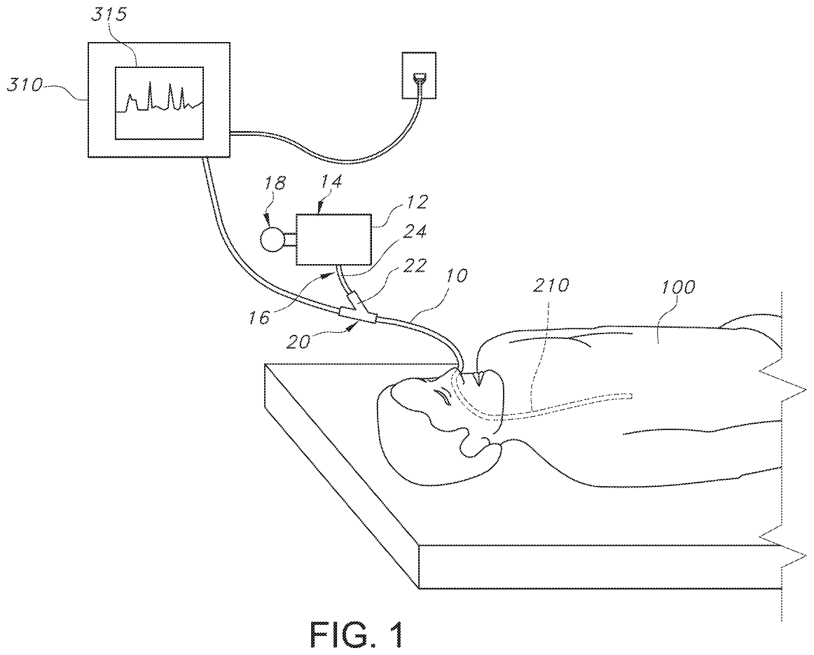

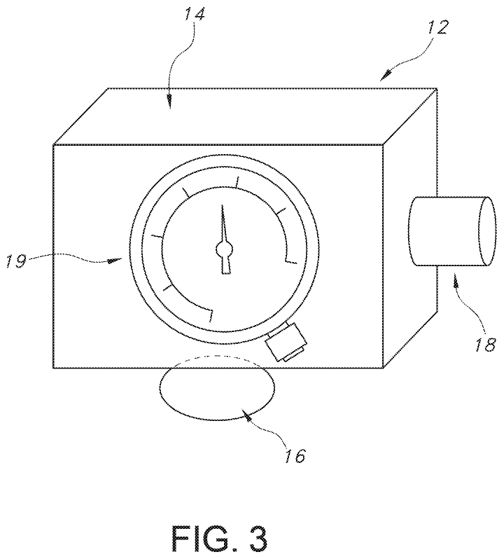

Particularly, the system of the present invention implements a differential air pressure sensor that is easily connected to a medical device, such as a catheter, and can be utilized by a healthcare provider to signal whether the medical device is in the patient's gastrointestinal tract or respiratory tract, thus confirming the position of the catheter in the patient's body. The specific features of the medical device position notification system of the present invention may be better understood with reference to FIGS. 1-5.

Referring now to FIG. 1, a medical device position notification system contemplated by the present invention includes: a medical device 10 and a differential air pressure sensor 12. The differential air pressure sensor 12 includes a housing 14, a first port 16 and a second port 18. The first port 16 is configured to be coupled to a connector 20 of the medical device, e.g., via a connection tube 24 that is connected through an access port 22 of the connector 20. Alternatively, the first port 16 and the access port 22 may be in direct fluid communication, e.g., wherein the first port 16 receives the access port 22 of the connector 20. The second port 18 is configured to be able to receive ambient, atmospheric air. The medical device 10 further includes a catheter 210, e.g., an enteral feeding tube, having an attachment end at a proximal end 214 coupled to the connector 20 and a distal end 212 configured to be inserted into a patient's body. The first port 16 may be removably secured to the connection tube 24 and/or the access port 22 via any acceptable means known in the art to secure a sealed, airtight fit. In certain embodiments, the differential air pressure sensor 12 may be permanently affixed to or permanently secured to the access port 22 of the connector 20. The catheter 210 further includes a distal end 212 that may be inserted into an orifice of a patient, i.e. the patient's nose, as shown in FIG. 1, or mouth. The distal end 212 of the catheter may be designed such that it can be guided down the esophagus into the patient's stomach. In some embodiments, it is contemplated that the differential air pressure sensor 12, connector 20, and catheter 210 may all be permanently secured, thus comprising one device without any removable parts. In some embodiments, it is contemplated that the differential air pressure sensor 12 may be removably attached to the medical device 10 via the connector 20.

As shown in FIGS. 1-3, the differential air pressure sensor 12 is configured to measure the air pressure within the catheter 210 in relation to the ambient atmospheric air pressure. Air pressure waves from the catheter 210 are received through the first port 16. The second port 18 is open to the ambient atmospheric air. The differential air pressure sensor 12 captures air pressure data received from the first port 16, which relates to the position of the medical device 10 within the patient's body 100. The differential air pressure sensor 12 may include a visual pressure gauge 19 on the exterior of the housing 14 that is configured to visually display the differential air pressure sensed from the catheter 210 in real time. The sensor 12 may be connected to a processor 310 via a wired or wireless connection. The sensor 12 may be located externally to the catheter 210, as shown in FIGS. 1-3, inside the catheter 210 (not shown), or within the processor 310 (not shown). The sensor 12 can send signals and communicates information about the differential air pressure within the catheter 210 with the processor 310. The processor 310 is configured to interpret the signals or information sent from the sensor 12. The processor 310 may be configured to use signal processing software to identify when the catheter 210 is improperly placed into the patient's airway. The processor 310 may be coupled to a display device 315, which displays the information relating to the position of the medical device 10 within the patient's body 100. The display device 315 may relay real time information regarding the differential air pressure in the catheter 210 during placement of the catheter 210 in the patient's body.

In some embodiments, e.g., as shown in FIG. 1, the display 315 displays the actual differential air pressure detected by the sensor 12 that is received by the processor 310. Additionally or alternatively, the display 315 may display a non-numerical visual indicator of the position of the catheter 210 within the patient's body, such as one or more colors, symbols, or words. As an example, the visual indicator may comprise a plurality of light emitting diodes (LEDs), such as green, yellow, and red LEDs. In such embodiments, the display 315 illuminates the green LED(s) if the sensed differential air pressure is generally constant and does not fluctuate in a pattern that mimics inhalation and exhalation, illuminates the yellow LED(s) if the differential air pressure is beginning to rise and fall in a pattern that mimics inhalation and exhalation, and illuminates the red LED(s) if the differential air pressure over time follows a sinusoidal rise and fall pattern above and below the ambient atmospheric pressure mimicking inhalation and exhalation. As another example, the display 315 may illuminate a series of colored or non-colored LEDs or display words or symbols to indicate various states of the sensed differential air pressure, e.g., a graphical representation as shown in FIGS. 1 and 4-5. In still other embodiments, the display 315 may utilize audible feedback, such as one or more beeping or other sounds, in addition to or in lieu of visual feedback to convey to the user of the system 100 the position of the medical device 10 as sensed by the differential air pressure sensor 12.

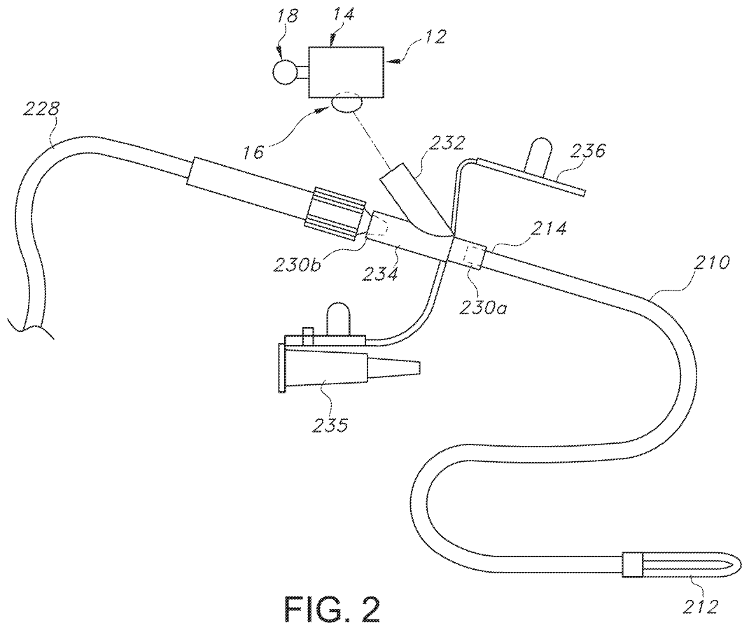

Turning now specifically to FIG. 2, in certain embodiments the medical device 10, may include a catheter, such as an enteral feeding tube 210. The enteral feeding tube 210 extends from a distal end 212 to a proximal end 214 and can be connected to a distal end 230a of a connector 230 at the proximal end 214 of the feeding tube 210. The connector 230 may also include a proximal end 230b. The connector 230 can extend along a longitudinal axis with a lumen 234 extending therebetween. Both the distal end 230a and proximal end 230b of the connector 230 can contain openings in communication with the lumen 234 and configured to receive the feeding tube 210. Optionally, the connector 230 can also include a cap or cover 235 configured to close the opening at the proximal end 230b of the connector 230. Optionally, the connector 230 can also include a cap or cover 235 configured to close the opening at the proximal end 230b of the connector 230. In addition, the connector 230 can include a Y-port 232 in communication with the lumen 234 and the opening at the distal end 230a. The Y-port 232 can additionally have a cap or cover 236 configured to close the opening at the proximal end 232a of the Y-port. The Y-port 232 can be configured to receive tubing or other suitable means for delivering enteral feeding fluid, medicine, or other fluids through the feeding tube 210. The medical device 10 may optionally include a tubing assembly 228 configured to connected to the connector 230 at the proximal end 230b of the connector. The tubing assembly 228 may be connected to a nutrition or medication source (not shown), and thus provide nutrition or medication through the connector 230 into the feeding tube 210, which ultimately provides nutrition and/or medication to the patient. The differential air pressure sensor 12 can be configured to be coupled to the Y-port 232 in order to sense the air pressure within the enteral feeding tube 210. In this manner, a healthcare provider can utilize readings from the differential air pressure sensor 12 in order to confirm the placement of the distal end 212 of the feeding tube 210. In some embodiments, the differential air pressure sensor 12 may be coupled to or attached to the connector 230 and left in place during delivery of nutrition or medication to the patient.

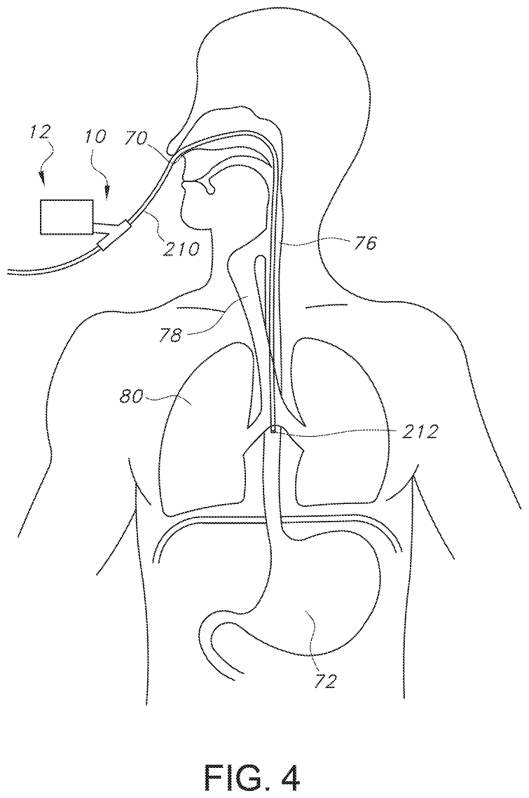

In certain embodiments, the differential air pressure sensor 12 may be used in conjunction with a variety of implantable medical device, e.g., catheter, applications. In one application illustrated in FIGS. 1 and 4, the differential air pressure sensor 12 is used in an enteral application. Here, a portion of the medical device 10, in this case an enteral catheter, is placed through an orifice 70 of the patient 100, such as the patient's nose or mouth. The enteral catheter may include a tube 210 that is inserted into the orifice 70 of the patient. The tube 210 includes a distal end 212 in order to deliver nutrition to the patient that may be positioned in the stomach 72 or small intestine 74 of the patient 100. The differential air pressure sensor 12 may be connected to an access port 232 of the tube 210. In other embodiments, the differential air pressure sensor 12 may can be connected directly to the catheter tube 210 (not shown). The distal end or tip 212 of the medical device 10 can ultimately be positioned in the stomach 72 or the small intestine 74. However, misplacement of the distal end 212 in the patient's respiratory tract, e.g., the trachea, bronchi, or lungs, rather than in the patient's gastrointestinal tract is a complication of insertion of enteral catheters due to the bifurcation of the esophagus 76 and the trachea 78. It is known that the bifurcation of the esophagus 76 and the trachea 78, as illustrated in FIG. 4, occurs at a certain distance from the entrance to the nostril in a patient 100, with the certain distance varying between pediatric and adult patients. Knowing this distance for a given patient, as well as the length of the enteral catheter tube 210, the user can determine how much (or what length) of the tube 210 has been inserted into the patient and, thus, know whether the distal end 212 of the tube 210 is at or near the point where the trachea branches off from the digestive tract, from which the tube 210 could be misplaced into the patient's airway. As an example, bifurcation typically occurs around 18-20 cm from the entrance to the nostril in adults; the area where bifurcation occurs may be referred to as a bifurcation zone.

Accordingly, as the health care provider advances the medical device 10 towards the patient's stomach 72 or when the health care provider believes they may be close to the bifurcation zone, they can initiate sensing with the differential air pressure sensor 12 to observe information related to the position of the distal end 212 in the patient's body 100. The location confirmation of the distal end 212 of the tube 210 can be made as follows: (1) if the differential air pressure is generally constant, the distal end 212 of the tube 210 is in the esophagus 76 and placement can continue through the digestive tract, but (2) if the differential air pressure rises and falls in a pattern that generally mimics inhalation and exhalation, e.g., in a sinusoidal pattern, the distal end 212 of the tube 210 is in the airway, e.g. the trachea 78 or lungs 80, and the tube 210 should be repositioned (see FIG. 4).

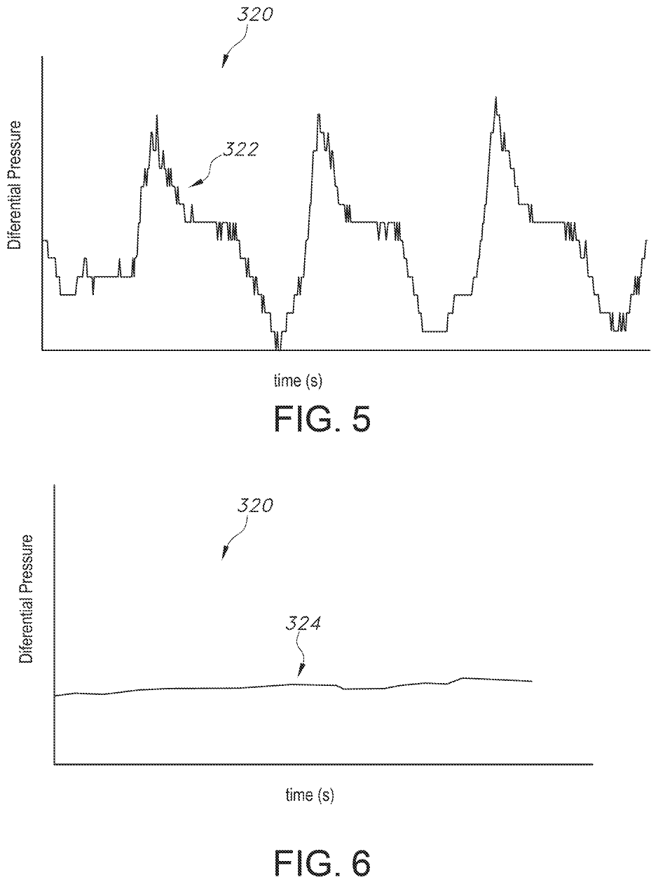

The differential air pressure sensor 12 is configured to continuously sense the air pressure within the tube 210 in relation to the ambient atmospheric pressure. As the tube 210 is advanced through the body, pressure readings detected by the sensor 12 may change based on the air pressure sensed at the distal end 212 of the tube 210. For example, when the distal end 212 of the tube 210 is in the airway, such as the trachea, the differential air pressure will fluctuate up and down in accordance with inspiration and exhalation, i.e., air entering and exiting the lungs. Whereas, if the distal end 212 of the tube 210 is in the esophagus, the differential air pressure will be generally constant. The display device 315 may provide information regarding the location of the distal end 212 of the tube 210, such as in the form of a graph 320. (See FIGS. 5 and 6.). The y-axis of the graph 320 corresponds to differential air pressure and the x-axis of the graph corresponds to time. Although, in other embodiments, the y-axis may correspond to time and the x-axis may correspond to differential air pressure (not shown). Accordingly, the graph 320 may illustrate the air pressure at the distal end 212 of the tube 210 in relation to ambient atmospheric pressure over time. As shown in FIG. 5, when the distal end of the tube 212 is in the trachea or respiratory tract, the graph 320 will begin showing fluctuations of differential air pressure following a generally sinusoidal pattern 322 that mimics the flow of breathing air in and out of the lungs. As shown in FIG. 6, however, when the distal end of the tube 212 is in the esophagus or gastrointestinal tract, the graph 320 will show a generally constant level 324 of differential air pressure. Accordingly, differentiating between these two signals allows for location identification of the distal end 212 of the tube 210 to be known in real time throughout the course of placing the tube 210 in the patient's body. Thus, the location of the distal end 212 of the tube 210 can be made as follows: (1) if the sensor 12 measures a generally constant differential air pressure within the tube 210, then the distal end 212 of the tube 210 is in the esophagus 76 and placement can continue through the digestive tract, but (2) if the sensor 12 measures fluctuations in the differential air pressure of the tube 210 mimicking a breathing pattern, the distal end 212 of the tube 210 is in the airway, e.g. the trachea 78 or lungs 80, and the tube 210 should be repositioned. Following placement confirmation of the tube 210, the differential air pressure sensor 12 may be removed from the tube 210.

In some embodiments, the medical device positioning system described herein may be used in conjunction with other suitable types of medical device monitoring systems, including but not limited to: carbon dioxide monitoring systems, light sensors, and sound sensors. Further, the differential air pressure sensor 12 described herein may be used in conjunction with other types of medical device position detectors, such as position detector signal generators, e.g. electromagnetic field generator systems, in order to confirm placement of the medical device in a patient's body. In these embodiments, while a signal generator may be utilized to communicate the placement of the medical device during and after insertion into the patient's body, the healthcare provide can utilize the differential air pressure sensor disclosed herein to confirm the placement of the medical device.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. A medical device position notification system comprising:
a medical device, wherein at least a portion of the medical device is configured to be inserted into a patient's body; and
a differential air pressure sensor, wherein the differential air pressure sensor is configured to provide information relating to the position of the medical device in the patient's body.

2. The medical device position notification system of claim 1, wherein the differential air pressure sensor is configured to measure air pressure within the medical device compared to ambient atmospheric air pressure.

3. The medical device position notification system of claim 2, wherein when the differential air pressure measured over time matches a pattern of inhalation and exhalation, the medical device is in the trachea or respiratory tract of the patient's body.

4. The medical device position notification system of claim 2, wherein when the differential air pressure measured over time does not match a pattern of inhalation and exhalation, the medical device is in the esophagus or gastrointestinal tract of the patient's body.

5. The medical device position notification system of claim 1, wherein the differential air pressure sensor comprises a first port for receiving air flow from the medical device and a second port for receiving air flow from ambient air.

6. The medical device position notification system of claim 1, wherein the differential air pressure sensor is configured to be electrically connected to at least one processor, wherein the differential air pressure sensor measures information relating to the position of the medical device within the patient's body and sends signals containing the information relating to the position of the medical device within the patient's body to the processor via a wired or wireless electrical connection in real-time, further wherein a display device is coupled to the processor and displays information relating to the position of the medical device within the patient's body communicated by the differential air pressure sensor.

7. The medical device position notification system of claim 1, wherein the medical device comprises a catheter.

8. The medical device position notification system of claim 7, wherein the catheter is configured to be inserted into at least one orifice the patient's body.

9. The medical device position notification system of claim 8, wherein the at least one orifice comprises a nose or a mouth.

10. The medical device position notification system of claim 7, wherein the differential air pressure sensor is coupled to the catheter via a connector.

11. A method for medical device position guidance comprising:
providing a medical device, wherein at least a portion of the medical device is configured to be inserted into the body;
providing a differential air pressure sensor, wherein the differential air pressure sensor is configured to be coupled to the medical device;
inserting the medical device into an orifice of the body;
activating the differential air pressure sensor to sense air pressure in the medical device compared to ambient atmospheric air pressure; and
observing the differential air pressure sensor or a display device configured to be coupled the differential air pressure sensor to determine the position of the medical device within the patient's body.

12. The method of claim 11, wherein the orifice is a nose or a mouth.

13. The method of claim 11, wherein the differential air pressure sensor is configured to measure the air pressure within the medical device compared to the ambient atmospheric air pressure.

14. The method of claim 13, wherein the observing step comprises observing the differential air pressure within the medical device over time, wherein if a pattern indicating inhalation and exhalation is observed, the medical device is in the trachea or respiratory tract of the patient's body.

15. The method of claim 11, wherein the observing step comprises observing the differential air pressure within the medical device over time, wherein if a pattern indicating inhalation and exhalation is not observed, the medical device is in the esophagus or gastrointestinal tract of the patient's body.

16. The method of claim 11, wherein the differential air pressure sensor is configured to be electrically connected to at least one processor, wherein the at least one differential air pressure sensor receives information relating to the position of the medical device within the patient's body and sends signals containing the information relating to the position of the medical device within the patient's body to the processor via a wired or wireless electrical connection in real-time, further wherein the display device is coupled to the processor and displays information relating to the position of the medical device within the patient's body communicated by the differential air pressure sensor.

17. The method of claim 11, wherein the medical device comprises a catheter.

18. The method of claim 17, wherein the differential air pressure sensor is coupled to the catheter via a connector.
